(19) United States
(12) Patent Application Publication
(10) Pub. No.: US 2012/0045486 A1
(43) Pub. Date: Feb. 23, 2012

(54) DELIVERY SYSTEMS

(75) Inventors: Marcelo Leonardo Bravo Cordero, Oxfordshire (GB); Huw Lyn Jones, Nottinghamshi (GB); Nazim Mohamed Kanji, Leicestershire (GB)

(73) Assignee: OXFORD PHARMASCIENCE LIMITED, CENTRE FOR INNOVATION & ENTERPRISE, Oxfordshire (GB)

(21) Appl. No.: 13/266,039

(22) PCT Filed: Apr. 23, 2010
(86) PCT No.: PCT/GB2010/050673

§ 371 Date: Oct. 24, 2011

(30) Foreign Application Priority Data
Apr. 24, 2009 (GB) 0907019.4

(51) Int. Cl. A61K 47/36; A61K 9/14

(52) U.S. Cl. 424/400; 514/777

(57) ABSTRACT

The present invention provides a delivery system for active and functional ingredients. In particular, the delivery systems of the present invention find particular application in the delivery of active and functional ingredients, such as medicaments, pharmaceuticals, nutritional supplements, botanicals, cosmeceuticals etc. Further, the invention relates to a delivery system for oral or topical administration of such active and functional ingredients. The invention is a delivery system based on a particulate gel precursor that acts both as the bodying agent,as well as the dispersing and suspending agent in the formulation. By modifying the level of precursor and process conditions, a broad range of product consistencies can be achieved ranging from thin liquid suspension to firm or semi solid gel. The precursor gel is a particulate linear chain fuctan gel. Inulin is a preferred fructan.

### CROSS-REFERENCE TO RELATED APPLICATIONS

This application is a United States national phase application under 35 U.S.C. §371 of International Patent Application No. PCT/GB2010/050673 filed on Apr. 23, 2010, and claims the benefit of Great Britain Patent Application No. 0907019.4 filed on Apr. 24, 2009, both of which are herein incorporated in their entirety by reference. The International Application was published as International Publication No. WO 2010/122357 A2 on Oct. 28, 2010.

### FIELD

The present invention relates to a delivery system for active and functional ingredients. In particular, the delivery systems of the present invention find particular application in the delivery of active and functional ingredients, such as medicaments, pharmaceuticals, nutritional supplements, botanicals, cosmeceuticals, etc. Further, the invention relates to a delivery system for oral or topical administration of such active and functional ingredients.

### BACKGROUND

There are many industries in which aqueous based delivery system are used to deliver active and functional ingredients. Ingredients may be added in a range of ways, such as small molecules, larger molecules, polymers, particles including colloids, etc. Where these ingredients are not water soluble, then arrays of further chemicals, such as dispersants, surfactants and thickeners (many of them synthetic) must be used to disperse and hold the ingredients in the system. Novel delivery systems, which can deliver hydrophobic or insoluble ingredients in aqueous medium are in demand.

One key area where such systems are used is for the delivery of compounds such as medicines, therapeutics, nutraceuticals, botanicals and cosmeceuticals. However, there are any other industries in which such systems can be used, including cosmetics, personal products including skin care, hair care and oral care products, detergents, paints, to name but a few.

There are a broad range of known delivery systems which are used as carriers for delivering drugs, medicaments and nutritional supplements. These include tablets, capsules, suspensions, gels, chews, lozenges, powders, etc. Children, the elderly and people with motor problems often have problems swallowing pills and capsules. So liquid or other fluid forms of delivery systems are preferred for these sectors of the population.

Pharmaceutical preparations in liquid and semi solid form for both oral administration and topical application are well know in the art. Aqueous based delivery systems, however, pose unique challenges, as many drugs and food supplements are hydrophobic. This makes them difficult to disperse and stabilise. Failure to achieve adequate dispersal and stability within delivery system, will affect the dosage which is delivered.

Conventional suspensions and gels use thickeners such as microcrystalline cellulose, carboxymethylcellulose, xanthan gum, gelatine and agar. They also use emulsifiers and/or surfactants such as polysorbate, sorbitans or others to disperse and suspend or emulsify hydrophobic ingredients. Typically these formulations require the thickening or gelling agent to be prepared separately from the dispersion or emulsification of the active ingredient. This adds complexity to the manufacturing process. Emulsions can be difficult to manufacture and can be unstable. Further they may also have inadequate viscosity.

When preparing formulations it is important to ensure compatibility of the formulation components with the active agents.

Also many drugs and supplements have taste profiles which can be unacceptable to consumers. Palatability to children and adults becomes an important consideration in a non-solid formulation for oral administration, as they can linger in the mouth. Certain medicinal ingredients, in addition to having an unpleasant taste, create a burning or scratching sensation in the throat, particularly the mucosa at the back of the throat when swallowed. This can be expressed through a throat catch or cough.

The taste profile can be masked, but this may frequently involve adding sugar, sugar alcohols and artificial sweeteners. Many children's medications for example will contain sugars to make them palatable, commonly in the form of sucrose, fructose or glucose syrup or a combination of sugar alcohols and artificial sweeteners. However, these are not components which are desirable for administration to children.

One example of a medicine which is prepared in liquid form for children is ibuprofen. This is a good example of the prior art. Ibuprofen is practically insoluble in water. It is a white crystalline, slightly waxy solid with a slight odour. It has a strong bitter taste and produces a burning sensation when in contact with the mucosa at the back of the throat. One approach to such a formulation is to suspend the finely divided ibuprofen in an aqueous medium with suspending agents and sweetening agents to mask the bitter taste of any dissolved ibuprofen. Ibuprofen's hydrophobic nature makes it difficult to disperse in water without the use of wetting agents such as polysorbates, which are conventional surfactants.

One such composition is described in U.S. Pat. No. 4,684,666 as a stabilised liquid ibuprofen syrup suitable for oral administration comprising 50 to 400 mg of ibuprofen per 5 ml of syrup, the ibuprofen being suspended in an aqueous liquid having more than 50% by weight of a polyhydric alcohol bodying agent, a sweetening agent and a pH of higher than 7.0 and below 7.7. Another such composition is described in U.S. Pat. No. 4,788,220 where the ibuprofen is maintained in suspension by a primary suspending agent such as xanthan gum, microcrystalline cellulose, sodium carboxymethyl cellulose or polysorbate 80, and where the ibuprofen is taste-masked with sucrose and/or sorbitol solution and the pH is maintained at about 3.5 to 5. Another earlier approach is to form a salt of ibuprofen with, for example, aluminium as described in U.S. Pat. No. 4,361,580. Such aluminium ibuprofen salts, which are essentially tasteless, are not soluble in water. They are also formulated with suspending agents and sweeteners.

### SUMMARY OF THE INVENTION

The present inventors have developed a delivery system based on a particulate gel precursor that acts both as the bodying agent, as well as the dispersing and suspending agent in the formulation. By modifying the level of precursor and process conditions, a broad range of product consistencies can be achieved ranging from thin liquid suspension to firm or semi solid gel. The particulate gel precursor exhibits outstanding wetting and dispersing properties and can disperse and suspend practically insoluble molecules, such as ibuprofen, without the need for emulsifiers or surfactants and keep them in stable suspension. This system achieves a substantially uniform and stable dispersion of the functional ingredient.

In pharmaceutical applications this means that ingredients, such as solid particles or molecules of ibuprofen, can be held in dispersion leading to faster rates of dissolution in in vitro testing. This also provides for minimal molecule diffusion helping, in oral preparations, to reduce contact with the mucosa at the back of the throat, thus eliminating or reducing “burn”.

The present inventors have discovered that linear chain fructan gels have particular properties which enable them disperse and suspend particles in this way.

Most plants store starch or sucrose as reserve carbohydrates, but about 15% of all flowering plant species store fructans, which are linear and branched polymers of fructose. Among the plants that store fructans are many of significant economic importance, such as cereals (e.g. barley, wheat, and oat), vegetables (e.g. chicory, onion, and lettuce), ornamentals (e.g. dahlia and tulip), and forage grasses (e.g. Lolium and Festuca) (Hendry and Wallace (1993) The origin, distribution, and evolutionary significance of fructans. In M Suzuki, N J Chatterton, eds, Science and Technology of Fructans. CRC Press, Boca Raton, Fla., pp 119-139). Fructans isolated from these plants have a variety of applications. Small fructans have a sweet taste, whereas longer fructan chains form emulsions with a fat-like texture and a neutral taste. The human digestive tract does not contain enzymes able to degrade fructans; therefore, there is strong interest from the food industry to use them as low-calorie food ingredients. In plants, fructans may have functions other than carbon storage; they have been implicated in protecting plants against water deficit caused by drought or low temperatures (Hendry and Wallace, 1993; Pilon-Smits et al, (1995) Improved performance of transgenic fructan-accumulating tobacco under drought stress. Plant Physiol 107: 125-130).

The present invention relates to linear chain fructans, including levans. Inulin is of particular interest.

Inulin is a naturally occurring storage polysaccharide present in numerous plants such as chicory root, wheat, asparagus, onions, garlic, dahlias, and Jerusalem artichoke. Chemically, inulin is a linear polydisperse fructan (degree of polymerisation (“DP”) 2-60 or higher) consisting of fructose molecules linked by β(2-1) glycosidic bonds with, generally, a terminal glucose unit connected to the last fructose with a α(1-2) bond. Several inulin types occur in nature and they differ in the degree of polymerisation and molecular weight, depending on the source, the harvest time, and processing conditions. Inulin has a mild sweet taste but it is not absorbed and does not affect blood sugar levels. It is widely used as an additive in the food industry e.g. a sweetener and stabiliser.

It is known to use inulin at relatively high concentrations (when compared to a typical hydrocolloid gel) in an aqueous liquid in order to form a gel (e.g. Kim, et al (2001), Factors Affecting Gel Formation of Inulin, Carbohydrate Polymers, 46, 135-145). For example, in a concentration of 15 to 35 weight % in water it forms generally after shearing a creamy structure which is in fact a specific gel network, namely a particulate gel. Inulin gels are generally described to be particulate gels composed of a tri-dimensional network of insoluble sub-micron crystalline particles with large amounts of water immobilised which assures its physical stability. Where the degree of polymerisation is ≦10, then inulin does not gel (see Chiavaro et al, (2007) Physiochemical characterization and stability of inulin gels, Eur Food Res Technol 225:85-94). At this chain length, the inulin can be classified as oligosaccharide.

Gels are defined as substantially dilute cross-linked systems, which exhibit no flow when in the steady-state. Gels can also be defined as an insoluble semi-rigid form of solid dispersion in a liquid. By weight, gels are mostly liquid, yet they behave like solids due to a three-dimensional cross-linked network within the liquid. It is the cross-links within the fluid that give a gel its structure (hardness) and contribute to stickiness.

Cross-linking polymers, carbohydrates or proteins are all ingredients that can be used for making gels. Generally, gels made from biopolymers can be classified into “associative” or “particulate” gels according to the mechanism of networking between polymer molecules (Clark, A J (1996) Biopolymer gels. Current Opinion in Colloids and Interface Science, 1(6), 712-717). In the associative case, random coils of polysaccharides undergo three dimensional transitions from coil to helix shape during gelation. This helps polymer chains form a network structure between molecules. Thermo-reversible gelations of polysaccharides (agar, carageenan and alginate) and fibrous protein are included in this category. The other type, the particulate gel, is made through large, random aggregation between polymer chains. The formation of gel from association of milk casein micelles (e.g. cheese, yoghurt) is included in this particulate gel type.

Inulin gels are used in the food industry where they are used as a fat replacer in table and dairy spreads, butter-like products, cream cheeses, milk drinks, yogurts and other products. Inulin particulate gels can be made from shearing or heating-cooling of an inulin suspension in water and the factors affecting gel formation of inulin are relatively well understood. Studies by Kim et al (ibid) established that the best range of conditions for gel formation are 20-30% (w/w) inulin concentration, 80-90° C. heating for 3-5 minutes at pH 6-8 and then cool down at room temperature. At severe conditions, such as high temperature or low pH's (90° C. or 100° C. and pH 1-2), inulin chains are hydrolised into smaller chains during heating and lead to non-gel forming components. At low concentrations (≦5%, w/v), inulin-water mixtures do not form a gel structure. While thermally induced gels show stronger gel strengths at the same concentration of inulin, shear induced gels at room temperature can also be made. This later technique provides for simpler, less costly processing and also minimises risk of hydrolysis of the inulin polymer. Gels formed by the shear induced process form gels with hydrogen bonds and Van der Waals interactions among inulin particles in dispersion while thermally induced inulin gels can form gels through entanglement of molecules. With very high shearing there are practically no differences between shear induced and thermally induced gels.

Specifically, the invention is based on using a specific particulate gel comprising a linear chain fructan, especially inulin, in water as a precursor in a fluid delivery system for active or functional ingredients. This finds particular application as a delivery system for pharmaceuticals, therapeutic, nutritional, botanical or cosmeceutical additives. The fructans used in the present invention are linear chain fructans having an average DP is greater than 10 and suitably where the average DP is equal or greater than 20, or even 25. Chicory inulin works particularly well in the present invention. Generally this has a DP of 2 to 65 DP, at least 17% having a DP of at least 40.

According to a first aspect of the present invention there is provided the use of a particulate linear chain fructan gel as a precursor for an aqueous delivery system for active and functional ingredients.

According to a second aspect of the present invention there is provided the use of a particulate linear chain fructan gel as an aqueous delivery system for active and functional ingredients.

According to a third aspect of the present invention there is provided the a precursor gel for an aqueous delivery system comprising a particulate linear chain fructan gel and one or more active or functional ingredients dispersed suspended or solubilised in the linear chain fructan gel.

According to a fourth aspect of the present invention there is provided an aqueous delivery system comprising a particulate linear chain fructan gel and one or more active or functional ingredients and, optionally, other excipients dispersed, suspended or solubilised in the linear chain fructan gel. The linear chain fructan gel enables active or functional ingredients to be dispersed, suspended or solubilised, even when the active or functional ingredients is not water soluble or is hydrophobic.

Further, the linear chain fructan gel precursor used in the present invention comprises 15% to 100% of linear chain fructan in water w/v %. Thicker gels are obtained by using more fructan. Suitable quantities of fructan are equal or greater than 25% w/v and equal or greater than 35% w/v of linear chain fructan in water. However, increasing fructan content increases cost. So preferred top ends of the range may be up to and including 75% w/v or even 60 % w/v. Linear chain fructan gels may be formed having 50% to 100% of fructan in water w/v %. (100% wt/v involves dissolving 1 kg fructan in 1 litre of water which gives a gel weighing 2 kg).

Further, the linear chain fructan gel used in the present invention is manufactured so that the average particle size of the gel suspended in water are on average between 70 μm to 1 μm, preferably between 30 μm to 1 μm, and ideally between 10 μm to 1 μm.

Particulate linear chain fructan gel precursors of this specification are then capable of dispersing and suspending or solubilising active ingredients and other excipients such as sweeteners, preservatives or colorants. For a pharmaceutical, therapeutic or nutritional ingredient, a typical range of addition would be to add up to 15 wt % of the finished product formulation. The present invention is based on the discovery that it is the particulate (rather than associative) gel nature of the system and the wetting nature of the fructan which enables the ingredients to be suspended within the system and this invention seeks to exploit that property. For ingested products, there can be rapid release of the active or functional ingredient when the system breaks open, e.g. in the stomach.

The linear chain fructan gel precursor formulation can then be further diluted in water or by other excipients such as sugar alcohols, non-digestible saccharides, such as oligofructose, or other syrups, where the fructan gel precursor can represent at least 25% of the finished formulation, depending on the desired finished product viscosity, texture and, optionally, taste. However, where the precursor is a thinner gel, then this level of dilution would be too high and the amount of fructan may fall below that which enables it to disperse and suspend the active or functional ingredient. So, the finished product should contain at least 10% w/w of linear chain fructan, and more preferably more than 15% w/w of linear chain fructan.

Thus, according to a fifth aspect of the present invention there is provided a composition comprising at least 25 wt % of a particulate linear gel chain fructan and up to 15 wt % of one or more active or functional ingredients dispersed, suspended or solubilised in the linear chain fructan gel and in which the linear chain fructan content is greater than 10% w/w.

The linear chain fructan gels of the present invention may be formed in a range for consistencies. If the product is to be used for topical application, then a thick gel will be preferred. If the product is for oral delivery of an active ingredient, then a syrup or liquid may be preferred.

Oligofructose is an ideal diluent, particularly where the linear chain fructan is inulin, given its compatibility with longer chain fructans and particularly inulin gels.

The term “non-digestible” means a substance which by virtue of its chemical structure is able to pass through the mouth and stomach substantially without change and is resistant to digestion by salivary and intestinal enzymes. Inulin is classed as a non-digestible polysaccharide.

Oligofructose or fructo-oligosaccharides is a subgroup of inulin, consisting of polymers with a degree of polymerisation (DP) ≦10. While oligofructose will not form gels given it is composed of molecules with a degree of polymerization below 10, it has a sweet, pleasant flavour and is substantially soluble in water at room temperature and the present inventors have discovered that it will work synergistically with the particulate gel system without affecting active ingredient dispersability and improving overall taste. Fructo-oligosaccharides are known to work synergistically with high-intensity artificial sweeteners, whose sweetness profile and aftertaste it, allowing for improved taste at reduced usage levels of artificial sweetener.

The system also exhibits excellent organoleptic properties. Mouth feel and rheology can be tuned but overall the system is pleasing on the mouth and does not linger in the mouth or throat.

The system is based on non digestible carbohydrates that have natural sweetness. Therefore there may be no need to include sugar, sugar alcohols or artificial sweeteners to mask the taste of an active functional ingredient. Where artificial sweeteners are included, the non-digestible carbohydrates work in synergy with the artificial sweeteners providing a pleasant background flavour. That favour can be further enhanced with specific taste masking technologies or flavouring components. Given the system is made from non-digestible polysaccharides it may impart additional health benefits.

Importantly, the inventors have also discovered that where this system is used to deliver for example 100 mg ibuprofen in a 5 ml dose formulation, then the “burn” at the back of the throat which might otherwise be expected, has been practically eliminated.

The method of manufacture can also be considerably simplified. The entire process can be implemented in a one batch process and at room temperature, where a mechanical shearing process is employed.

All the known art for the manufacture of fructan gels, and particularly of inulin gels, can be used to make a gel which is used in this invention. Particulate gel precursors can be manufactured via both thermal and mechanical shearing processes.

Typical embodiments exemplified in this specification use linear chain fructan particulate gels in a concentration of 50% to 100% w/v in water manufactured via a shearing process and these are further diluted in water or by other ingredients representing up to 75% of the finished formulation depending on the desired finished product viscosity, texture and taste.

The particulate gel precursor can provide body and can also wet, disperse and keep in stable suspension hydrophobic ingredients. Products made via this technology exhibit substantial uniform and stable dispersion of the functional ingredient without the need conventional dispersing and suspending ingredients, such as thickening gums, surfactants and emulsifiers, and so enable formula simplification.

However, the particulate gel precursor can also work in synergy with compatible polysaccharides and hydrocolloids to provide enhanced organoleptic and formulation properties. It is also known in the art that inulin gels, in particular, work synergistically with certain hydrocolloids such as starch, modified starch, dextrin, gelatin, gellan gum, etc.

Where the invention is being used as an oral delivery system, then conceivably, the specific beneficial ingredient which may be delivered through the oral delivery system of the present invention can be any one of the many pharmaceutical agents, therapeutic substances or nutritional substances that may be delivered orally. Some of these may also then be absorbed through the digestive tract and into the bloodstream. Examples of these include pharmaceutical agents, minerals, mineral sources, vitamins, vitamin sources, herbal extracts, botanical extracts and nutraceutical ingredients.

The active or functional ingredients useful herein can be selected from a large group of therapeutic agents. Respective classes include those in the following therapeutic categories: ace-inhibitors; alkaloids; antacids; analgesics; anabolic agents; anti-anginal drugs; anti-allergy agents; anti-arrhythmia agents; antiasthmatics; antibiotics; anticholesterolemics; anticonvulsants; anticoagulants; antidepressants; antidiarrheal preparations; anti-emetics; antihistamines; antihypertensives; anti-infectives; anti-inflammatories; antilipid agents; antimanics; anti-migraine agents; antinauseants; antipsychotics; antistroke agents; antithyroid preparations; anabolic drugs; antiobesity agents; antiparasitics; antipsychotics; antipyretics; antispasmodics; antithrombotics; antitumor agents; antitussives; antiulcer agents; anti-uricemic agents; anxiolytic agents; appetite stimulants; appetite suppressants; beta-blocking agents; bronchodilators; cardiovascular agents; cerebral dilators; chelating agents; cholecystekinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypoglycemic agents; ion-exchange resins; laxatives; migraine treatments; mineral supplements; mucolytics, narcotics; neuroleptics; neuromuscular drugs; non-steroidal anti-inflammatories (NSAIDs); nutritional additives; peripheral vasodilators; polypeptides; prostaglandins; psychotropics; renin inhibitors; respiratory stimulants; sedatives; steroids; stimulants; sympatholytics; thyroid preparations; tranquilizers; uterine relaxants; vaginal preparations; vasoconstrictors; vasodilators; vertigo agents; vitamins; wound healing agents; and others. However, these are examples and are not limiting of the application of the present invention. The only criteria as to whether the drug would be useful in the delivery system is whether it can provide its therapeutic effect after ingestion and its compatibility with the matrix. Other criteria to consider is the drug's dissolution rate, shelf life stability and taste.

Although the present invention may be used to deliver a drug active or functional ingredient which is water soluble, it may also be used to deliver those which are not water soluble. It may be particularly effective for delivering many unpleasant tasting actives or functional ingredients currently available on the Rx and over-the-counter market. Non-limiting examples of some of the types of actives or functional ingredients mentioned above include include: acetaminophen; acetic acid; acetylsalicylic acid, including its buffered forms; acrivastine; albuterol and its sulfate; alcohol; alkaline phosphatase; allantoin; aloe; aluminum acetate, carbonate, chlorohydrate and hydroxide; alprozolam; amino acids; aminobenzoic acid; amoxicillin; ampicillin; amsacrine; amsalog; anethole; ascorbic acid; aspartame; astemizole; atenolol; azatidine and its maleate; bacitracin; balsam peru; BCNU (carmustine); beclomethasone diproprionate; benzocaine; benzoic acid; benzophenones; benzoyl peroxide; benzquinamide and its hydrochloride; bethanechol; biotin; bisacodyl; bismuth subsalicylate; bornyl acetate; bromopheniramine and its maleate; buspirone; caffeine; calamine; calcium carbonate, casinate and hydroxide; camphor; captopril; cascara sagrada; castor oil; cefaclor; cefadroxil; cephalexin; centrizine and its hydrochloride; cetyl alcohol; cetylpyridinium chloride; chelated minerals; chloramphenicol; chlorcyclizine hydrochloride; chlorhexidine gluconate; chloroxylenol; chloropentostatin; chlorpheniramine and its maleates and tannates; chlorpromazine; cholestyramine resin; choline bitartrate; chondrogenic stimulating protein; cimetidine and its hydrochloride; cinnamedrine hydrochloride; citalopram; citric acid; clarithromycin; clemastine and its fumarate; clonidine and its hydrochloride salt; clorfibrate; cocoa butter; cod liver oil; codeine and its fumarate and phosphate; cortisone acetate; ciprofloxacin HCl; cyanocobalamin; cyclizine hydrochloride; cyproheptadine and its hydrochloride; danthron; dexbromopheniramine maleate; dextromethorphan and its hydrohalides; diazepam; dibucaine; dichloralphenazone; diclofen and its alkali metal salts; diclofenac sodium; digoxin; dihydroergotamine and its hydrogenates/mesylates; diltiazem; dimethicone; dioxybenzone; diphenhydramine and its citrate; diphenhydramine and its hydrochloride; divalproex and its alkali metal salts; docusate calcium, potassium, and sodium; doxycycline hydrate; doxylamine succinate; dronabinol; efaroxan; enalapril; enoxacin; ergotamine and its tartrate; erythromycin; estropipate; ethinyl estradiol; ephedrine; epinephrine bitartrate; erythropoietin; eucalyptol; famotidine; fenoprofen and its metal salts; ferrous fumarate, gluconate and sulfate; fluoxetine; folic acid; fosphenytoin; 5-fluorouracil (5-FU); fluoxetine and its hydrochloride; flurbiprofen; furosemide; gabapentan; gentamicin; gemfibrozil; glipizide; glycerine; glyceryl stearate; granisetron and its hydrochloride; griseofulvin; growth hormone; guafenesin; hexylresorcinol; hydrochlorothiazide; hydrocodone and its tartrates; hydrocortisone and its acetate; 8-hydroxyquinoline sulfate; hydroxyzine and its pamoate and hydrochloride salts; ibuprofen; indomethacin; inositol; insulin; iodine; ipecac; iron; isosorbide and its mono- and dinitrates; isoxicam; ketamine; kaolin; ketoprofen; lactic acid; lanolin; lecithin; leuprolide acetate; lidocaine and its hydrochloride salt; lifinopril; liotrix; loratadine; lovastatin; luteinizing hormore; LHRH (lutenizing hormone replacement hormone); magnesium carbonate, hydroxide, salicylate, and trisilicate; meclizine and its hydrochloride; mefenamic acid; meclofenamic acid; meclofenamate sodium; medroxyprogesterone acetate; methenamine mandelate; menthol; meperidine hydrochloride; metaproterenol sulfate; methscopolamine and its nitrates; methsergide and its maleate; methyl nicotinate; methyl salicylate; methyl cellulose; methsuximide; metoclopramide and its halides/hydrates; metronidazole and its hydrochloride; metoprotol tartrate; miconazole nitrate; mineral oil; minoxidil; morphine; naproxen and its alkali metal sodium salts; nifedipine; neomycin sulfate; niacin; niacinamide; nicotine; nicotinamide; nimesulide; nitroglycerine; nonoxynol-9; norethindrone and its acetate; nystatin; octoxynol; octoxynol-9; octyl dimethyl PABA; octyl methoxycinnamate; omega-3 polyunsaturated fatty acids; omeprazole; ondansetron and its hydrochloride; oxolinic acid; oxybenzone; oxtriphylline; para-aminobenzoic acid (PABA); padimate-O; paramethadione; pentastatin; peppermint oil; pentaerythritol tetranitrate; pentobarbital sodium; perphenazine; phenelzine sulfate; phenindamine and its tartrate; pheniramine maleate; phenobarbital; phenol; phenolphthalein; phenylephrine and its tannates and hydrochlorides; phenylpropanolamine and its hydrochloride salt; phenytoin; pirmenol; piroxicam and its salts; polymicin B sulfate; potassium chloride and nitrate; prazepam; procainamide hydrochloride; procaterol; promethazine and its hydrochloride; propoxyphene and its hydrochloride and napsylate; pramiracetin; pramoxine and its hydrochloride salt; prochlorperazine and its maleate; propanolol and its hydrochloride; promethazine and its hydrochloride; propanolol; pseudoephedrine and its sulfates and hydrochlorides; pyridoxine; pyrolamine and its hydrochlorides and tannates; quinapril; quinidine gluconate and sulfate; quinestrol; ralitoline; ranitadine; resorcinol; riboflavin; salicylic acid; scopolamine; sesame oil; shark liver oil; simethicone; sodium bicarbonate, citrate, and fluoride; sodium monofluorophosphate; sucralfate; sulfanethoxazole; sulfasalazine; sulfur; sumatriptan and its succinate; tacrine and its hydrochloride; theophylline; terfenadine; thiethylperazine and its maleate; timolol and its maleate; thioperidone; tramadol; trimetrexate; triazolam; tretinoin; tetracycline hydrochloride; tolmetin; tolnaftate; triclosan; trimethobenzamide and its hydrochloride; tripelennamine and its hydrochloride; tripolidine hydrochloride; undecylenic acid; vancomycin; verapamil HCl; vidaribine phosphate; vitamins A, B, C, D, B₁, B₂, B₆, B₁₂, E, and K; witch hazel; xylometazoline hydrochloride; zinc; zinc sulfate; zinc undecylenate. Mixtures and pharmaceutically acceptable salts of these and other actives can be used.

The delivery system is particularly useful for active agents which are sparingly soluble solid agents whose dissolution and release properties may be enhanced by the dispersing nature of the composition. These agents include H₂ antagonists, analgesics, including non-steroidal anti-inflammatory drugs (NSAIDs), anticholesterolemics, anti-allergy agents, and anti-migraine agents.

Analgesics include aspirin, acetaminophen, acetaminophen plus caffeine, and non-steroidal anti-inflammatory drugs (NSAIDS), e.g., ibuprofen and nimesulide, NSAIDs include ibuprofen; diclofenac and its alkali metal salts; fenoprofen and its metal salts; flurbiprofen; ketoprofen; naproxen and its alkali metal salts; nimesulide; and piroxicam and its salts; H₂-antagonists include cimetidine, ranitidine hydrochloride, famotidine, nizatidine, ebrotidine, mifentidine, roxatidine, pisatidine and aceroxatidine; anti-allergy agents include hydricodone and its tartrates; clemastine and its fumarate; azatadine and its maleate; acetaminophen; hydroxyzine and its pamoate and hydrochloride salts; chlorpheniramine and its maleates and tannates; pseudoephedrine and its sulfates and hydrochlorides; bromopheniramine and its maleate; dextromethorphan and its hydrohalides; loratadine; phenylephrine and its tannates and hydrochlorides; methscopolamine and its nitrates; phenylpropanolamine and its hydrochlorides; codeine and its hydrochloride; codeine and its phosphate; terfenadine; acrivastine; astemizole; cetrizine and its hydrochloride; phenindamine and its tartrate; tripelennamine and its hydrochloride; cyproheptadine and its hydrochloride; promethazine and its hydrochloride; and pyrilamine and its hydrochlorides and tannates; antimigraine agents include divalproex and its alkali metal salts; timolol and its maleate; propanolol and its hydrohalides; ergotamine and its tartrate; caffeine; sumatriptan and its succinate; dihydroergotamine, its hydrogenates/mesylates; methsergide and its maleate; isometheptene mucate; and dichloralphenazone.

Another class of drugs which could be used are antiemetics. Useful antiemetics include: meclizine and its hydrochloride; hydroxyzine and its hydrochloride and pamoate; diphenhydramine and its hydrochloride; prochlorperazine and its maleate; benzquinamide and its hydrochloride; granisetron and its hydrochloride; dronabinol; bismuth subsalicylate; promethazine and its hydrochloride; metoclopramide and its halides/hydrates; chlorpromazine; trimethobenzamide and its hydrochloride; thiethylperazine and its maleate; scopolamine; perphenazine; and ondansetron and its hydrochloride.

Other active ingredients which could be delivered by the present invention include antidiarrheals such as immodium AD, antihistamines, antitussives, decongestants, vitamins, and breath freshners. Also contemplated for use herein are anxiolytics such as Xanax; antipsychotics such as Clozaril and Haldon; antihistamines such as Seldane, Hismanal, Relafen, and Tavist; antiemetics such as Kytril and Cesamet; bronchodilators such as Bentolin, Proventil; antidepressants such as Prozac, Zoloft, and Paxil; antimigranes such as Imigran, ACE-inhibitors such as Vasotec, Capoten and Zestril; Anti-Alzheimers agents such as Nicergoline; and Ca^{II}-Antagonists such as Procardia, Adalat, and Calan and anticholesterolemics, including statins, such as atorvastatin, fluvastatin, lovastatin, provastatin, mevastatin, pitavastatin, simvastatin and the like.

Nutritional functional ingredients which may be delivered by a the delivery system according to the present invention include (but are not limited to) coated omega3, acerola, beta-carotene, bioflavonoids, boron, brewer's yeast, chondroitin sulphate, chromium, cranberry extract, evening primrose oil, folic acid, garlic, germanium, glucosamine sulphate, gingko biloba, ginseng, guarana, phosphorous, plant sterols, safflower oil, selenium, silicon, soya extract and wheat germ oil, and a number of botanical extracts.

Where the invention is being used as a topical delivery system, then suitable pharmaceutically active agents may be any drug substance capable of exerting a desired therapeutic or prophylactic effect at the site of application or following uptake through the skin, e.g. an antibiotic, antiinflammatory or antipruritic effect. Many if not most of the drug substances applied topically in conventional topical compositions (e.g. steroids, NSAIDs (for example ibuprofen), antifungals (for example ketoconazole), lithium compounds (for example for treating sebnorrhaic dermatitis or molluscum contagiosum), anti-acne compounds (for example azelaic acid), anti-dandruff agents (such as zinc pyrithione), etc.) may be used in the delivery systems of the invention. This includes those identified above in relation to oral delivery systems, which could also be delivered topically, especially those which could be delivered through topical applications in the mouth. In the case of the cosmetic compositions, the cosmetically active agent may be any substance capable of exerting a desired cosmetic effect at the site of application or following uptake into the skin, e.g. vitamins, cosmeceuticals, plant oils, UV absorbers, skin hydrating agents, cleansing agents, colorants, aromas, etc. Once again, many if not most cosmetic agents applied topically in conventional topical compositions may be used in the delivery systems of the invention. Such active agents may be used in concentrations similar or comparable to the currently used concentrations.

Combinations of various types of active or functional ingredient, as well as combinations of individual active or functional ingredient, are contemplated.

By way of example, ibuprofen suspension products made with an inulin particulate gel precursor and complemented with oligofructose show significant improved taste and reduced back of the throat burn as against the leading commercial paediatric formulation.

The formulation may also include other excipients such as preservatives, colourants and flavours. Although, the formulation does not require dispersants, surfactants or emulsifiers, such additives are not excluded.

According to a further aspect of the invention there is provided a method of making the precursor gels discussed above comprising the steps of (1) mixing linear chain fructans and water to form a gel and (2) dispersing, suspending or solubilising one or more active or functional ingredients and, optionally, other excipients, in the fructans gel.

According to a further aspect of the invention there is provided a method of making the aqueous delivery system and compositions discussed above comprising diluting the precursor gels discussed above with acceptable excipients.

The technology can be implemented via a simple one batch process, such as the following:
1 First preservatives or sweeteners are dissolved in purified water in the main vessel.
2 The linear chain fructan, such as inulin, is then added slowly to the main vessel whilst homogenising until well dispersed. Inulin forms a thick white gel. The gel may be formed using a mechanical high shear process or a process which uses a low shear together with heating and cooling of the gel. The selection of method depends on what is to be suspended and how much gel is to be used in the final product. Where a mechanical high shear process is employed, then the manufacture can take place at room temperature.
3 More purified water is added and homogenisation continues to form a thick white liquid.
4 The active or functional ingredient is then added slowly to the main vessel whilst stirring in order to disperse it into the bulk liquid. Some such ingredients (e.g. ibuprofen) may need to be sieved to break up lumps.
5 Diluents, such as oligofrutose, are then added whilst stirring.
6 Flavouring and any other ingredients may be added at this point.
7 Further purified water is added to adjust volume and viscosity, and the mixture stirred further.

### BRIEF DESCRIPTION OF THE EMBODIMENTS

Specific embodiments of the invention will now be described by way of example.

In the embodiments, the following commercially sourced components were used, unless stated otherwise:

| | |
|---|---|
| Inulin | Orafti ®GR |
| Oligofructose | Orafti ®L95 |
| Maltitol syrup | Lycasin ® 80/55 |

In the embodiments viscosity was measured using Brookfield DV-E viscometer, spindle 3@12 rpm.

Base System for Ibuprofen Suspension 100 mg/5 ml (“Base System”)

The findings of optimisation trials resulted in the following base formula and manufacturing process (2 litre batch size), to which additional materials such as preservatives and flavours could be incorporated.

| Material | mg/5 ml | Weight (g) - 2 L batch |
|---|---|---|
| Ibuprofen | 100 | 40 |
| Inulin | 1250 | 500 |
| Oligofructose | 1480 | 592 |
| Sodium Saccharin | 5 | 2 |
| Purified Water | To 5 ml | To 2000 ml |

1. Dissolve sodium saccharin (2 g) in 400 g purified water.
2. Add 500 g inulin slowly to the main vessel whilst homogenising until well dispersed and a thick white gel is formed.
3. Add 300 g purified water and continue to homogenise, forming a thick white liquid
4. Sieve ibuprofen to break up lumps and add slowly to the main vessel whilst stirring in order to disperse into the bulk liquid.
5. Add oligofructose (592 g) whilst stirring.
6. Make up to volume with purified water and continue to stir.

This Base System was used to manufacture a number of concepts (examples 1-7 inclusive). These were evaluated for initial viscosity and taste. Viscosity on two month old samples stored in laboratory conditions was also measured. Selected samples were also evaluated for ibuprofen dissolution using the USP method for Ibuprofen Oral Suspensions and compared with commercial samples of leading commercially available ibuprofen products designed for children.

| | | Examples 3-5, 7 | Example 6 |
|---|---|---|---|
| | Example 1 | Various | High |
| | Base Ibuprofen | Embodiments | Viscosity |
| Gel precursor | | | |
| Water in precursor | 700 | 700 | 800 |
| Inulin | 500 | 500 | 800 |
| Actives | 40 | 40 | 40 |
| FOS/syrups | 592 | 592 | 592 |
| Other ingredients | 2 | 12 | 2 |
| Additional water | 550 | 550 | 105 |
| % inulin in water in | 71.4% | 71.4% | 100.0% |
| precursor (% w/v) | | | |
| % precursor gel in | 35.00% | 35.00% | 40.00% |
| finished product | | | |
| (% w/w) | | | |

### EXAMPLE 1

Objective—to manufacture a product according to the Base System and process

| Material | mg/5 ml | Weight (g) - 2 L batch |
|---|---|---|
| Ibuprofen | 100 | 40 |
| Inulin | 1250 | 500 |
| Oligofructose | 1480 | 592 |
| Sodium Saccharin | 5 | 2 |
| Purified Water | To 5 ml | To 2000 ml |

| Process | Key observations & actions |
|---|---|
| Dissolve sodium saccharin (2 g) in 400 g | — |
| purified water. | |
| Add 500 g inulin slowly to the main vessel | Thick, white gel formed |
| whilst homogenising until well dispersed | |
| and a thick white gel is formed. | |
| Add 300 g purified water and continue to | |
| homogenise | |
| Sieve ibuprofen through a 850 μm sieve to | Ibuprofen dispersed readily |
| break up lumps and add slowly to the main | in the thick liquid at low |
| vessel whilst stirring in order to disperse | stir speed without the need |
| into the bulk liquid. | to homogenise |
| Add oligofructose (592 g) whilst stirring. | |
| Make up to volume with purified water and | 550.2 g added V = 1290 |
| stir | cps |
| | +1 day V = 3370 cps, |
| | ibuprofen well dispersed |
| | +2 months V = 1710 cps*, |
| | ibuprofen well dispersed |

V = viscosity (cps) measured using Brookfield DV-E viscometer, spindle 3 @ 12 rpm
*Pungent odour suggesting sample may have deteriorated due to absence of preservatives

### Taste

The batch was tasted following manufacture. It had a sweet, smooth mouthfeel. The taste/throat burn of ibuprofen was well masked.

### Dissolution vs Leading Commercial Product

A dissolution test was performed to investigate the rate at which the ibuprofen is made available in solution at low pH. In vitro dissolution rate tests were conducted using the dissolution method for ibuprofen oral suspensions (USP 2009, Vol II) and comparing the performance of Example 1 versus an off-the-shelf sample of a leading paediatric product.

Mean Ibuprofen Dissolution (%)

| Time (mins) | Example 1 | Commercial product |
|---|---|---|
| 0 | 0 | 0 |
| 15 | 96 | 75 |
| 30 | 96 | 84 |
| 45 | 95 | 84 |
| 60 | 92 | 88 |

As can be seen from the above table, after 15 minutes, Example 1 shows more dissolution than the commercial product, with 96% of the ibuprofen in Example 1 dissolved in that time, versus only 75% for the commercial product. Importantly, the system achieves repeatable results within a tight range when compared to highly variable results for the commercial product.

These results confirm that Example 1 could be capable of delivering a fast dissolution rate for ibuprofen and has significantly less variability of dissolution rate than that shown by a current leading commercial product. This is due to the outstanding dispersing and wetting properties of the system of the present invention.

### EXAMPLE 2

Objective—to manufacture a product according to the Base System and process, modified by use of inulin Orafti® ST grade in place of GR grade.

| Material | mg/5 ml | Weight (g) - 2 L batch |
|---|---|---|
| Ibuprofen | 100 | 40 |
| Inulin | 1250 | 500 |
| Oligofructose | 1480 | 592 |
| Sodium Saccharin | 5 | 2 |
| Purified Water | To 5 ml | To 2000 ml |

| Process | Key observations & actions |
|---|---|
| Dissolve sodium saccharin (2 g) in 400 g | — |
| purified water. | |
| Add 500 g inulin slowly to the main vessel | Thick, white gel formed |
| whilst homogenising until well dispersed | |
| and a thick white gel is formed. | |
| Add 300 g purified water and continue to | |
| homogenise | |
| Sieve ibuprofen through a 850 μm sieve to | Ibuprofen dispersed readily |
| break up lumps and add slowly to the main | in the thick liquid at low |
| vessel whilst stirring in order to disperse | stir speed without the need |
| into the bulk liquid. | to homogenise |
| Add oligofructose (592 g) whilst stirring. | |
| Make up to volume with purified water and | 626.8 g added |
| stir | +1 day V = 3300 cps, |
| | good dispersion |
| | +2 months V = 4340 cps, |
| | good dispersion |

V = viscosity (cps) measured using Brookfield DV-E viscometer, spindle 3 @ 12 rpm

### Taste

Sweet, smooth mouthfeel, very good masking of the ibuprofen taste/throat burn

### Dissolution vs Leading Commercial Product

In vitro dissolution rate tests were conducted using the dissolution method for ibuprofen oral suspensions (USP 2009, Vol II) and comparing the performance of Example 2 versus an off-the-shelf sample of a leading paediatric product.

Mean Ibuprofen Dissolution (%)

| Time (mins) | Example 2 | Commercial product |
|---|---|---|
| 0 | 0 | 0 |
| 15 | 93 | 75 |
| 30 | 92 | 84 |
| 45 | 90 | 84 |
| 60 | 91 | 88 |

### EXAMPLE 3

Objective—To manufacture a product according to the Base System and process, modified by inclusion of citric acid monohydrate 0.5%.

| Material | mg/5 ml | Weight (g) - 2 L batch |
|---|---|---|
| Ibuprofen | 100 | 40 |
| Inulin | 1250 | 500 |
| Oligofructose | 1480 | 592 |
| Sodium Saccharin | 5 | 2 |
| Citric Acid Monohydrate | 25 | 10 |
| Purified Water | To 5 ml | To 2000 ml |

| Process | Key observations & actions |
|---|---|
| Dissolve sodium saccharin (2 g) and citric | — |
| acid monohydrate (10 g) in 400 g purified | |
| water. | |
| Add 500 g inulin slowly to the main vessel | Thick, white gel formed |
| whilst homogenising until well dispersed | |
| and a thick white gel is formed. | |
| Add 300 g purified water and continue to | |
| homogenise | |
| Sieve ibuprofen through a 850 μm sieve to | Ibuprofen dispersed readily |
| break up lumps and add slowly to the main | in the thick liquid at low |
| vessel whilst stirring in order to disperse | stir speed without the need |
| into the bulk liquid. | to homogenise |
| Add oligofructose (592 g) whilst stirring. | |
| Make up to volume with purified water and | 530.7 g added, pH = 2.4 |
| stir | +1 day V = 4600 cps, |
| | good dispersion |
| | +2 months V = 3500 |
| | cps, good dispersion |

V = viscosity (cps) measured using Brookfield DV-E viscometer, spindle 3 @ 12 rpm

### Taste

Sharper taste, smooth mouthfeel, very good masking of the ibuprofen taste/throat burn

### Dissolution vs Leading Commercial Product

In vitro dissolution rate tests were conducted using the dissolution method for ibuprofen oral suspensions (USP 2009, Vol II) and comparing the performance of Example 3 versus an off-the-shelf sample of a leading paediatric product.

Mean Ibuprofen Dissolution (%)

| Time (mins) | Example 3 | Commercial product |
|---|---|---|
| 0 | 0 | 0 |
| 15 | 98 | 75 |
| 30 | 96 | 84 |
| 45 | 94 | 84 |
| 60 | 92 | 88 |

### EXAMPLE 4

Objective—To manufacture a product according to the Base System and process, modified by inclusion of xanthan gum 0.5%.

| Material | mg/5 ml | Weight (g) - 2 L batch |
|---|---|---|
| Ibuprofen | 100 | 40 |
| inulin | 1250 | 500 |
| Oligofructose | 1480 | 592 |
| Sodium Saccharin | 5 | 2 |
| Xanthan Gum | 25 | 10 |
| Purified Water | To 5 ml | To 2000 ml |

| Process | Key observations & actions |
|---|---|
| Dissolve sodium saccharin (2 g) in 400 g | — |
| purified water. | |
| Add 500 g inulin slowly to the main vessel | Thick, white gel formed |
| whilst homogenising until well dispersed | |
| and a thick white gel is formed. | |
| Add 300 g purified water and continue to | |
| homogenise | |
| Sieve ibuprofen through a 850 μm sieve to | Ibuprofen dispersed readily |
| break up lumps and add slowly to the main | in the thick liquid at low |
| vessel whilst stirring in order to disperse | stir speed without the need |
| into the bulk liquid. | to homogenise |
| In separate vessel, to oligofructose (592 g) | Xanthan gum gel appears |
| mix 10 g Xanthan gum. Add to main vessel. | streaky within the white |
| Rinse vessel with 100 g purified water and | liquid, suggesting that |
| add to main vessel whilst stirring. | associative gels may not be |
| | compatible with particulate |
| | gels. |
| Make up to volume with purified water and | 480.9 g added |
| stir | +1 day V = 4060 |
| | cps, good dispersion of |
| | ibuprofen |
| | +2 months V = 6010 |
| | cps, good dispersion |

V = viscosity (cps) measured using Brookfield DV-E viscometer, spindle 3 @ 12 rpm

### Taste

Sweet, smooth mouthfeel, ibuprofen throat burn more pronounced

### Dissolution

Not tested

### EXAMPLE 5

Objective—to manufacture a product according to the Base System and process: replace oligofructose with maltitol liquid.

| Material | mg/5 ml | Weight (g) - 2 L batch |
|---|---|---|
| Ibuprofen | 100 | 40 |
| inulin | 1250 | 500 |
| Maltitol syrup | 1480 | 592 |
| Sodium Saccharin | 5 | 2 |
| Purified Water | To 5ml | To 2000 ml |

| Process | Key observations & actions |
|---|---|
| Dissolve sodium saccharin (2 g) in 400 g | — |
| purified water. | |
| Add 500 g inulin slowly to the main vessel | Thick, white gel formed |
| whilst homogenising until well dispersed | |
| and a thick white gel is formed. | |
| Add 300 g purified water and continue to | |
| homogenise | |
| Sieve ibuprofen through a 850 μm sieve to | Ibuprofen dispersed readily |
| break up lumps and add slowly to the main | in the thick liquid at low |
| vessel whilst stirring in order to disperse | stir speed without the need |
| into the bulk liquid. | to homogenise |
| Add maltitol syrup (592 g) whilst stirring. | |
| Make up to volume with purified water and | 579.1 g added V = <1000 |
| stir | cps - Thin liquid with |
| | ibuprofen on the surface |
| | +2 months V = 2550 cps |

V = viscosity (cps) measured using Brookfield DV-E viscometer, spindle 3 @ 12 rpm

### Taste

Sweet, smooth mouthfeel, ibuprofen throat burn more apparent. The product when made with maltitol produces less throat burn than commercially available products. So the outcome is an improvement over known products. However, the throat burn is more apparent with maltitol than with oligofructose, making maltitol a is less desired diluent than oligofructose.

### Dissolution

Not tested

### EXAMPLE 6

Objective—to manufacture a product according to the Base System and process: but modify by using a higher level of inulin to form a higher viscosity liquid

| Material | mg/5 ml | Weight (g) - 2 L batch |
|---|---|---|
| Ibuprofen | 100 | 40 |
| Inulin | 1500 | 800 |
| Oligofructose | 1480 | 592 |
| Sodium Saccharin | 5 | 2 |
| Purified Water | To 5 ml | To 2000 ml |

| Process | Key observations & actions |
|---|---|
| Dissolve sodium saccharin (2 g) in 800 g | — |
| purified water. | |
| Add 800 g inulin slowly to the main vessel | Thick, white gel formed |
| whilst homogenising until well dispersed | |
| and a thick white gel is formed. | |
| Sieve ibuprofen through a 850 μm sieve to | Ibuprofen dispersed readily |
| break up lumps and add slowly to the main | in the thick liquid at low |
| vessel whilst stirring in order to disperse | stir speed without the need |
| into the bulk liquid. | to homogenise |
| Add oligofructose (592 g) whilst stirring. | |
| Make up to volume with purified water and | 104.7 g added V = 1290 |
| stir | cps |
| | +1 day V = 18360 cps |
| | +2 months V = not tested - |
| | could not pour liquid out of |
| | bottle |

V = viscosity (cps) measured using Brookfield DV-E viscometer, spindle 3 @ 6 rpm

### Taste

Sweet, thick gel that ‘coats’ the buccal cavity, very good masking of the ibuprofen taste/throat burn

### Dissolution vs Leading Commercial Product

In vitro dissolution rate tests were conducted using the dissolution method for ibuprofen oral suspensions (USP 2009, Vol II) and comparing the performance of Example 6 versus an off-the-shelf sample of a leading paediatric product.

Mean Ibuprofen Dissolution (%)

| Time (mins) | Example 6 | Commercial product |
|---|---|---|
| 0 | 0 | 0 |
| 15 | 84 | 75 |
| 30 | 93 | 84 |
| 45 | 94 | 84 |
| 60 | 91 | 88 |

### EXAMPLE 7

Objective—to manufacture a product according to the Base System and process: modified by making up to volume with oligofructose instead of purified water

| Material | mg/5 ml | Weight (g) - 2 L batch |
|---|---|---|
| Ibuprofen | 100 | 40 |
| Inulin | 1250 | 500 |
| Oligofructose | To 5 ml | To 2000 ml |
| Sodium Saccharin | 5 | 2 |
| Purified Water | 1.75 | 700 |

| Process | Key observations & actions |
|---|---|
| Dissolve sodium saccharin | — |
| (2 g) in 400 g purified water. | |
| Add 500 g inulin slowly to the | Thick, white gel formed |
| main vessel whilst homogenising | |
| until well dispersed | |
| and a thick white gel is formed. | |
| Add 300 g purified water | |
| and continue to homogenise | |
| Sieve ibuprofen through a 850 μm | Ibuprofen dispersed readily in the |
| sieve to break up lumps and add | thick liquid at low stir speed |
| slowly to the main vessel whilst | without the need to homogenise |
| stirring in order to disperse | |
| into the bulk liquid. | |
| Make up to volume with | 1426.2 g added |
| oligofructose and stir | +1 day V = 5160 cps |
| | +2 months V = 5500 cps |

V = viscosity (cps) measured using Brookfield DV-E viscometer, spindle 3 @ 12 rpm

### Taste

Very sweet, smooth mouthfeel, very good masking of the ibuprofen taste/throat burn

### Dissolution vs Leading Commercial Product

In vitro dissolution rate tests were conducted using the dissolution method for ibuprofen oral suspensions (USP 2009, Vol II) and comparing the performance of Example 7 versus an off-the-shelf sample of a leading paediatric product.

Mean Ibuprofen Dissolution (%)

| Time (mins) | Example 7 | Commercial product |
|---|---|---|
| 0 | 0 | 0 |
| 15 | 87 | 75 |
| 30 | 91 | 84 |
| 45 | 89 | 84 |
| 60 | 87 | 88 |

### EXAMPLES 8-10

Ibuprofen 100 mg/5 ml Suspensions—Fully Preserved and Flavoured Samples

### Composition Details

The ibuprofen product can also be made with preservatives and flavours.

| | | | | Batch |
|---|---|---|---|---|
| | Example 8 | Example 9 | Example 10 | Weight |
| Material | (mg/5 ml) | (mg/5 ml) | (mg/5 ml) | (g) |
| Ibuprofen | 100 | 100 | 100 | 40 |
| Inulin | 1250 | 1250 | 1250 | 500 |
| Oligofructose | 1480 | 1480 | 1480 | 592 |
| Sodium Saccharin | 5 | 5 | 5 | 2 |
| Methyl parabens | 10 | 10 | 10 | 4 |
| (preservative) | | | | |
| Propyl parabens | 1 | 1 | 1 | 0.4 |
| (preservative) | | | | |
| Orange Flavour | — | 12.5 | — | 5 |
| Strawberry Flavour | — | — | 12.5 | 5 |
| Purified Water | to 5 ml | to 5 ml | to 5 ml | to 2000 ml |

### Method of Manufacture

1. Dissolve the methyl and propyl parabens in 400 g purified water (60-70° C.).
2. Add and dissolve the sodium saccharin (2 g).
3. Add 500 g inulin slowly to the main vessel whilst homogenising using a high shear mixer until a thick, white gel is formed.
4. Add 300 g purified water and continue to homogenise until a consistent liquid is produced.
5. Sift ibuprofen through a wire mesh to break up lumps and add slowly to the main vessel whilst stirring.
6. Add the oligofructose and continue to stir.
7. Add flavour whilst stirring.
8. Make up to volume (2000 ml) with purified water and stir.

### Taste

These products were sweet had a smooth mouthfeel. They were, very good masking of the ibuprofen taste/throat burn. These were compared to a leading commercial product of similar flavour (or no flavour). The embodiments of the invention were found to be significantly superior to the corresponding commercial product, particularly in terms of masking of the ibuprofen taste/throat burn

This process was also used to manufacture paracetamol and bismuth subsalicylate suspensions (composition details below).

### EXAMPLE 11

### Paracetamol 120 mg/5 ml Suspension

The Base System was then adapted to manufacture a paracetamol suspension, but this was not optimised for the change of active ingredient.

| | Example 11 | Batch Weight |
|---|---|---|
| Material | (mg/5 ml) | (g) |
| Paracetamol | 120 | 48 |
| Inulin | 1250 | 500 |
| Oligofructose | 1480 | 592 |
| Sodium Saccharin | 5 | 2 |
| Methyl parabens | 10 | 4 |
| Propyl parabens | 1 | 0.4 |
| Strawberry Flavour | 12.5 | 5 |
| Purified Water | to 5 ml | To 2000 ml |

| Appearance | |
|---|---|
| Initial | White, thin liquid, paracetamol was well dispersed |
| +1 day | White, thicker, smoother, paracetamol remained well |
| | dispersed |
| pH | 4.47 |
| Viscosity (cps) | |
| Initial | <1000 |
| +1 day | 1650 |

### EXAMPLE 12

### Bismuth Subsalicylate 87.6 mg/5 ml Suspension

The Base System was then adapted to manufacture a bismuth subsalicylate suspension, but this was not optimised for the change of active ingredient.

| | Example 12 | Batch Weight |
|---|---|---|
| Material | (mg/5 ml) | (g) |
| Bismuth subsalicylate | 87.6 | 17.52 |
| Inulin | 1250 | 250 |
| Oligofructose | 1480 | 296 |
| Sodium Saccharin | 5 | 1 |
| Methyl parabens | 10 | 2 |
| Propyl parabens | 1 | 0.2 |
| Purified Water | to 5 ml | To 1000 ml |

| Appearance | |
|---|---|
| Initial | Brilliant white, thin liquid, BS was well dispersed |
| +1 day | Brilliant white, thicker, smoother, BS remained well |
| | dispersed |
| pH | 3.87 |
| Viscosity (cps) | |
| Initial | <1000 |
| +1 day | 2300 |

### EXAMPLE 13

### Chesty Cough Liquid

The Base System was then adapted to manufacture a chesty cough liquid, but this was not optimised for the change of active ingredients.

Objective—Chesty Cough Liquid (Guaifenesin 100 mg/5 ml, Phenylephrine HCl 5 mg/5 ml, Levomenthol 1.1 mg/5 ml)

| Material | mg/5 ml | Weight (g) - 1 L batch |
|---|---|---|
| Guaifenesin | 100 | 20 |
| Phenylephrine HCl | 5 | 1 |
| Levomenthol | 1.1 | 0.22 |
| Inulin | 1250 | 250 |
| Oligofructose | 1480 | 296 |
| Sodium Saccharin | 5 | 1 |
| Methyl parabens | 10 | 2 |
| Propyl parabens | 1 | 0.2 |
| Cherry flavour | 12.5 | 2.5 |
| Purified Water | To 5 ml | To 1000 ml |

| Process | Key observations & actions |
|---|---|
| Dissolve the methyl and propyl parabens in | — |
| 400 g purified water (60-70° C.) | |
| Add and dissolve the sodium saccharin | |
| Add 250 g inulin whilst homogenising using | Thick white gel formed |
| a high shear mixer until a thick, white | |
| gel is formed | |
| In a separate vessel add 150 g purified | |
| water and dissolve the phenylephrine HCl. | |
| Add the solution to the main vessel whilst | |
| homogenising | |
| Sift guaifenesin through a wire mesh to | Guaifenesin dispersed read- |
| break up the lumps and add slowly to the | ily in the thick liquid at |
| main vessel whilst stirring | low stir speed without the |
| | need to homogenise |
| Add oligofructose whilst stirring. | |
| Dissolve levomenthol in the cherry flavour | |
| and add to the main vessel whilst stirring | |
| Make up to volume with purified water and | |
| stir | |
| Appearance | |
| Initial | White, thin liquid, |
| | guaifenesin was well |
| | dispersed |
| +1 day | White, thicker, smoother. |
| | Guaifenesin remained well |
| | dispersed |
| Taste | Smooth mouthfeel, bitter |
| | taste - system needed to be |
| | optimised to improve taste |
| pH | 3.199 |
| Viscosity (cps) | |
| Initial | <1000 |
| +1 day | 1870 |

V = viscosity (cps) measured using Brookfield DV-E viscometer, spindle 3 @ 12 rpm

### EXAMPLE 14

### Cold and Flu Product

The Base System was then adapted to manufacture a cold & flu product, but this was not optimised for the change of active ingredients.

Objective—Cold & Flu Liquid to match Vicks Dayquil (Paracetamol 325 mg/15 ml, Dextromethorphan HBr 10 mg/15 ml, Phenylephrine HCl 5 mg/15 ml)

| Material | mg/15 ml | Weight (g) - 1 L batch |
|---|---|---|
| Paracetamol | 325 | 21.67 |
| Dextromethorphan HBr | 10 | 0.67 |
| Phenylephrine HCl | 5 | 0.33 |
| Inulin | 3750 | 250 |
| Oligofructose | 4440 | 296 |
| Sodium Saccharin | 15 | 1 |
| Methyl parabens | 30 | 2 |
| Propyl parabens | 3 | 0.2 |
| Cherry flavour | 37.5 | 2.5 |
| Purified Water | To 15 ml | To 1000 ml |

| Process | Key observations & actions |
|---|---|
| Dissolve the methyl and propyl parabens in | — |
| 400 g purified water (60-70° C.) | |
| Add and dissolve the sodium saccharin | |
| Add 250 g inulin whilst homogenising using | Thick white gel formed |
| a high shear mixer until a thick, white gel is | |
| formed | |
| In a separate vessel add 150 g purified | |
| water and dissolve the phenylephrine HCl | |
| and dextromethorphan HBr. Add the | |
| solution to the main vessel whilst | |
| homogenising | |
| Sift paracetamol through a wire mesh to | Paracetamol dispersed read- |
| break up the lumps and add slowly to the | ily in the thick liquid at |
| main vessel whilst stirring | low stir speed without the |
| | need to homogenise |
| Add oligofructose whilst stirring. | |
| Add flavour whilst stirring | |
| Make up to volume with purified water and | |
| stir | |
| Appearance | |
| Initial | White liquid, paracetamol |
| | was well dispersed |
| +1 day | White, thicker, smoother. |
| | Paracetamol remained well |
| | dispersed |
| Taste | Smooth mouthfeel, bitter |
| | taste - system needed to |
| | be optimised to improve |
| | taste |
| pH | 3.199 |
| Viscosity (cps) | |
| Initial | <1000 |
| +1 day | 1950 |

V = viscosity (cps) measured using Brookfield DV-E viscometer, spindle 3 @ 12 rpm

### EXAMPLE 15

### Further Embodiments

Example 15 is an example of a further first series of embodiments of use of an inulin particulate gel precursor in a pharmaceutical, therapeutic or nutritional composition, the composition including a delivery system for a pharmaceutical, therapeutic, nutritional botanical or cosmeceutical functional ingredient.

### Example 15

Ibuprofen Suspension 100 mg/5 ml Formulation

| Ingredient | % w/w |
|---|---|
| Ibuprofen | 2 |
| Inulin | 30 |
| Maltitol solution | 15.0 |
| Glycerol | 10 |
| Xanthan Gum | 0.7 |
| Sodium Saccharin | 0.2 |
| Citric Acid | 0.5 |
| Sodium Citrate | 0.5 |
| Polysorbate 80 | 0.2 |
| Methyl Paraben | 0.2 |
| Propyl Paraben | 0.05 |
| Strawberry Flavour | 0.05 |
| Purified water to | 100 |

The following table shows examples of parameters of delivery systems in yet a further series of embodiments of the invention. In these embodiments, the delivery system includes inulin particulate gel precursors in combination with oligofructose.

Example delivery system parameters
for a further series of embodiments

| Ingredient | wt % |
|---|---|
| Fructo-oligo saccharide (FOS) | 5-80 |
| Inulin | 10-70 |
| Polydextrose | 0-70 |
| Other ingredients | 0-15 |
| Water | Up to 60 |
| Ratio inulin:FOS (dry) | at least 0.4, <3 |

### EXAMPLES 16-20

The following table shows specific examples of pharmaceutical, therapeutic, nutritional, botanical or cosmeceutical compositions according to the invention. In example 16, the delivery system is utilised in combination with a functional ingredient in the form of ibuprofen to provide an ibuprofen gel. Examples 16 to 20 include pharmaceutical functional ingredients such as ibuprofen, paracetamol, and dextromethorphan HBR.

Examples 18, 19 and 20 include relatively large amounts of added water, which provides a composition in the form of a suspension or liquid. The inventors have found that the delivery systems of the invention maintain the suspension of functional ingredients such as ibuprofen and paracetamol in water without the addition of conventional suspending agents used in suspension formulations, such as xanthan gum. Moreover, the delivery system is capable of wetting and dispersing hydrophobic functional ingredients such as ibuprofen in water without the use of conventional wetting agents such as surfactants. Since the wetting of hydrophobic molecules can be a rate limiting step for dissolution and absorption in the gastro intestinal tract, it is thought that the delivery systems of the invention have potential for increasing the rate of absorption and bioavailability of poorly soluble molecules.

### Example 16-20

### Pharmaceutical, Medicinal or Nutritional Compositions

All percentages are by weight w/w

| | Example | | | | |
|---|---|---|---|---|---|
| | 16 | 17 | 18 | 19 | 20 |
| Ingredient | % | % | % | % | % |
| Delivery | | | | | |
| system | | | | | |
| Inulin | 41.69 | 34.84 | 34.80 | 34.80 | 31.00 |
| Fructo- | 23.72 | 19.83 | 19.80 | 19.80 | 18.00 |
| oligosaccharide | | | | | |
| (FOS) syrup | | | | | |
| (25% water) | | | | | |
| Fructo- | 17.79 | 14.87 | 14.85 | 14.85 | 13.50 |
| oligosaccharide | | | | | |
| (FOS) dry basis | | | | | |
| or powder | | | | | |
| Polydextrose | 1.69 | 1.42 | 1.40 | 1.40 | 1.30 |
| Glycerin | | | | | 10.00 |
| Sodium saccharin | | | 0.20 | 0.20 | 0.20 |
| Sodium benzoate | | | | | 0.30 |
| Citric acid | | | | 0.50 | |
| Sodium citrate | | | | 0.50 | |
| Methyl paraben | | | 0.22 | 0.20 | |
| Propyl paraben | | | | 0.05 | |
| Flavour | | | 0.05 | 0.05 | 0.05 |
| Syrup water | 5.93 | 4.96 | 4.95 | 4.95 | 4.50 |
| Added water | 28.83 | 42.49 | 41.13 | 40.50 | 39.00 |
| Total water | 34.76 | 47.45 | 46.08 | 45.45 | 43.50 |
| Sub total | 95.93 | 98.58 | 97.60 | 98.00 | 99.85 |
| delivery system | | | | | |
| Functional | | | | | |
| ingredients | | | | | |
| Ibuprofen | 4.07 | 1.42 | | 2.00 | |
| Paracetamol | | | 2.40 | | |
| Dextro- | | | | | 0.15 |
| methorphan HBr | | | | | |
| Sub total | 4.07 | 1.42 | 2.40 | 2.00 | 0.15 |
| functional | | | | | |
| ingredients | | | | | |
| Total composition | 100 | 100 | 100 | 100 | 100 |
| Ratio delivery | 23.57 | 69.42 | 40.67 | 49.00 | 665.67 |
| system:functional | | | | | |
| ingredients | | | | | |
| Physical | Gel | Paediatric | Suspen- | Suspen- | Liquid |
| characteristics | | suspension | sion | sion | |

From the above examples, it can be seen that the invention provides an aqueous gel delivery system with improved organoleptic properties, particularly when made from inulin. The delivery system of the invention enables active or functional ingredients to be dispersed, suspended or solubilised and is able to provide a product which has advantages over commercially available alternatives and that this can be achieved without the use of the traditional surfactants and dispersants of the prior art. The embodiments are given are by way of example only and not intended to be limiting. Those skilled in the art will readily appreciate from the teachings in this specification how to make further embodiments.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. An aqueous system for delivering a pharmaceutical or a therapeutic agent comprising a particulate linear chain fructan gel and one or more pharmaceutical or therapeutic agents dispersed, suspended or solubilised in the linear chain fructan gel.

49. An aqueous delivery system according to claim 48 comprising at least 25 wt% of an linear chain fructan gel and wherein the linear chain fructan content of the delivery system is greater than 10% w/w.

50. An aqueous delivery system according to claim 48 wherein the linear chain fructan gel comprises a linear chain fructan having an average degree of polymerisation of more than 10.

51. An aqueous delivery system as claimed in claim 50 wherein the linear chain fructan has an average degree of polymerisation of more than 20.

52. An aqueous delivery system as claimed in claim 48 wherein the linear chain fructan is inulin having an average degree of polymerisation of more than 10 or chicory inulin, having a degree of polymerisation of between 2 and 60 and wherein at least 17% has a degree of polymerisation of at least 40.

53. An aqueous delivery system as claimed in claim 48 wherein average size of the particles forming the particulate gel are between 70 μm to 1 μm.

54. An aqueous delivery system as claimed in claim 48 comprising up to 15 wt % of the pharmaceutical or therapeutic agents.

55. An aqueous delivery system as claimed in claim 54 wherein the pharmaceutical or therapeutic agent is non-water soluble.

56. An aqueous delivery system as claimed in claim 55 wherein the pharmaceutical or therapeutic agent is hydrophobic.

57. An aqueous delivery system as claimed in any of claim 48 further comprising one or more acceptable excipients.

58. An aqueous delivery system as claimed in claim 57 where the one or more excipients are selected from sugar alcohols, non-digestible saccharides, and other pharmaceutically acceptable syrups.

59. An aqueous delivery system as claimed in claim 57 wherein the excipients include oligofructose.

60. A method of making an aqueous delivery system as claimed in any of claim 48 comprising the steps of (1) mixing linear chain fructan and water to form a linear chain fructan gel, and (2) dispersing, suspending or solubilising one or more pharmaceutical or therapeutic agents in the linear chain fructan gel.

61. A method of making an aqueous delivery system as claimed in claim 60 comprising the step of diluting the gel with acceptable excipients.

62. A method of making an aqueous delivery system as claimed in claim 60 wherein the aqueous delivery system comprises at least 25 wt% of an linear chain fructan gel and wherein the linear chain fructan content of the delivery system is greater than 10% w/w.

63. A method of making an aqueous delivery system as claimed in claim 60 wherein the linear chain fructan has having an average degree of polymerisation of more than 10.

64. A method of making an aqueous delivery system as claimed in claim 60 wherein the linear chain fructan is inulin having an average degree of polymerisation of more than 10 or chicory inulin, having a degree of polymerisation of between 2 and 60 and wherein at least 17% has a degree of polymerisation of at least 40.

65. A method of making an aqueous delivery system as claimed in claim 60 wherein the aqueous delivery system comprises at least 15 wt % of the pharmaceutical or therapeutic agents.

66. A method of making an aqueous delivery system as claimed in claim 60 wherein the pharmaceutical or therapeutic agent is non-water soluble.

67. A method of making an aqueous delivery system as claimed in claim 60 wherein one or more excipients are selected from sugar alcohols, non-digestible saccharides, and other pharmaceutically acceptable syrups are mixed into the gel.
